(19) United States
(12) Patent Application Publication
(10) Pub. No.: US 2026/0199263 A1
(43) Pub. Date: Jul. 16, 2026

(54) Composition For The Modified Release Of Biological Polyamines, In Particular Spermidine

(71) Applicant: GIULIANI S.P.A., Milano (IT)

(72) Inventors: Giammaria GIULIANI, Montagnola (CH); Fabio RINALDI, Milano (IT); Daniela PINTO, Milano (IT); Antonio MASCOLO, Milano (IT); Barbara MARZANI, Carbonara Al Ticino (IT)

(21) Appl. No.: 19/136,000

(22) PCT Filed: Dec. 5, 2023
(86) PCT No.: PCT/IB2023/062221

§ 371 Date: Jun. 5, 2025

(30) Foreign Application Priority Data
Dec. 6, 2022 (IT) 102022000025077

(51) Int. Cl. A61K 31/132; A61K 9/20; A61K 9/48

(52) U.S. Cl. CPC A61K 31/132; A61K 9/2013; A61K 9/4858

(57) ABSTRACT

The invention relates to a composition for the modified release of a biologically active aliphatic polyamine comprising palmitoylethanolamide and preferably a sucroester as agents for modifying the release of the aliphatic polyamine from the composition. The composition can be used as is or added to the formulation of a supplement to promote hair growth.

### CAMPO DELL'INVENZIONE

The invention relates to a composition for the modified release of biologically active aliphatic polyamines, in particular spermidine or a salt thereof.

The present invention originates in the pharmaceutical sector, in particular in the field of matrices or systems for the controlled release of biologically active ingredients.

### BACKGROUND OF THE INVENTION

Oral administration allows suitable blood levels of the active ingredient to be obtained in a relatively short time. Formulations of drugs intended for oral administration are made in such a way as to maintain the blood levels of the active ingredient below the toxic threshold and above the concentration at which they are ineffective, for the longest possible period of time.

This aim can be achieved by using specific administration or dosage regimes or by using particular forms of administration.

One of these consists in the administration of multiple doses throughout the day.

This approach has limitations, mainly represented by low compliance and unsuitable blood profiles. Systems for the controlled release of active ingredients have been developed to overcome these drawbacks.

Controlled release formulations serve the purpose of reducing the frequency of administration of drugs with a short half-life or short-lasting effect.

These formulations also limit fluctuations in plasma drug concentration, helping to provide a more uniform therapeutic effect and minimizing adverse effects. These formulations also make it possible to increase tolerability and compliance of the treated subject towards the drug, thanks to the reduction in the frequency of drug administration, and to improve effectiveness since a high percentage of patients achieve a greater therapeutic response by resorting to the administration of reduced drug dosages for a prolonged period of time.

Modified release formulations regulate the release of the active ingredient from the carrier by controlling its release rate, thus maintaining the active ingredient plasma levels within the range of therapeutic efficacy.

Different types of forms for the modified release of active ingredients are known.

One of these is represented by enteric release forms, designed to resist the acidic pH of the stomach's gastric juices, to reduce the release of the active ingredient within the stomach and release it into an environment where higher pH values, generally comprised between 5.5 and 6.5, typical of the small intestine and colon, are present.

Other forms of administration involve coating the active ingredient with waxy substances or substances with low water solubility, typically including the drug in a matrix that slowly releases it during transit through the gastrointestinal tract or by complexing the drug with polymers, such as ion exchange resins.

The variables that influence the release of the active ingredients present in these systems are multiple and linked to the matrix polymer type choice, the multiple chemical-physical characteristics of the active ingredients, and also of the excipients present in the formulation.

Other factors, such as permeability, shape and size of the oral formulation, influence the release kinetics of the active ingredient too.

To modulate the release of an active ingredient, it is also possible to bring active ingredients that are poorly water soluble into contact with highly soluble substances, in order to favor solubilization or dispersion of the active ingredients in the aqueous medium. Substances commonly used for these purposes are alcohols, polyalcohols, amphiphilic substances, such as surfactants, or mixtures of substances which develop effervescence in contact with water, such as mixtures of acids and carbonates or bicarbonates.

The use of pharmaceutical technologies and equipment allows modifications to the active substances release profile to be obtained. However, the use of complementary formulation technologies causes an increase in the complexity of the study required for the formulation with a consequent increase in costs and development times.

Among substances endowed with biological activity, there is growing interest in aliphatic polyamines, in particular spermidine. This molecule is a natural inducer of autophagy and has a variety of beneficial effects on health, including anti-tumor, neuromodifying, anti-aging, anti-inflammatory, cardiovascular system protection activities, etc. The most recent scientific research also suggests that foods rich in spermidine may be used to prevent diseases related to the aging of the organism.

It is currently believed that increasing the amount of luminal polyamines in the colon promotes human longevity, similarly to what has been demonstrated in animal models.

There is therefore an interest in promoting the integration of biogenic polyamines of interest, in particular spermidine, into the dietary regime.

A suitable supplementation of these amines can be achieved by increasing the consumption of foods that contain spermidine or by taking compositions or supplements in which it is present in physiologically acceptable amounts.

The absorption of aliphatic polyamines occurs in humans mainly in the upper part of the intestine; however, biogenic amines can pass from the lumen into the bloodstream even at the level of the distal part of the digestive system, through the colon mucosa.

However, it has been found that the intake of biologically active polyamines through the diet is often inadequate or lacking, particularly in subjects who follow a vegan/vegetarian dietary pattern or lifestyle.

Furthermore, the formulations of supplements containing spermidine currently on the market are not suitable for reaching and maintaining an adequate plasma concentration over time, or require multiple daily administrations to achieve the desired effects.

The same applies to multicomponent formulations (with multiple functional substances or active ingredients) of matrix systems, which do not allow spermidine to be released according to the desired kinetics without influencing the release of the other substances of physiological interest present in the formulation.

Similarly, there is a need to have dosage forms that modulate the release of biogenic amines, guaranteeing the release of said substances also in the distal part of the digestive system, and specifically at the colon level where it can be integrated with the bacterial flora making up the physiological human microbiota.

At present, there is therefore a need to have systems suitable for modulating rate, time and anatomical area for the release of biogenic polyamines, such as spermidine and derivatives thereof, with the aim of achieving physiological or therapeutic objectives.

One of the objects of the present invention consists in providing a formulation that allows the controlled release of biogenic polyamines, in particular spermidine, in order to achieve and maintain the desired plasma levels for a prolonged period of time to achieve physiological or therapeutic objectives.

Another object of the invention consists in providing a composition for the prolonged release of biogenic aliphatic polyamines which is simple to implement and does not involve high production costs.

### DESCRIPTION OF THE INVENTION

The inventors have unexpectedly found that the formulation of aliphatic polyamines with palmitoylethanolamide, and preferably a sucroester, achieves the effect of reducing the frequency of administration and releasing therapeutically effective amounts of polyamines both in the proximal portion of the small intestine and in the distal part of the large intestine, specifically at the colon level. The release of exogenous polyamines in the colon portion of the intestine supplements the endogenous polyamines deriving from the metabolic activity of the intestinal microbiota which regulates multiple biological processes in the host.

These effects correspond to a stimulating activity on the physiological growth process of hair and skin adnexa, such as beard, eyelashes, eyebrows and nails. A cosmetic or therapeutic effect on the keratin structures of the body such as hair, eyelashes, eyebrows, nails and beard, and an effect on their growth regardless of the etiology of their loss are thus achieved.

According to a first aspect, the object of the present invention is a composition for the modified release of a biologically active aliphatic polyamine comprising a biologically active aliphatic polyamine, at least a substance for modifying the release of the polyamine, and a physiologically acceptable carrier, wherein the substance for modifying the release includes palmitoylethanolamide.

Suitable aliphatic polyamines include putrescine, spermine, spermidine, and mixtures thereof. According to a preferred embodiment, the polyamine is spermidine or a salt thereof, preferably spermidine hydrochloride.

According to a preferred and advantageous embodiment, the modified release composition according to any one of the embodiments further described herein, further comprises a sucroester as a substance for modifying the release of spermidine.

Advantageously, the ratios between palmitoylethanolamide/spermidine, palmitoylethanolamide-spermidine and sucroester, and also the sucroester HLB, can be varied in order to modulate the kinetics of spermidine release from the modified release composition described herein.

The palmitoylethanolamide present in the composition described herein modifies the polyamine release along the digestive tract and increases the intestinal absorption of the polyamine, particularly in the colon region.

In another aspect, the invention concerns a use of palmitoylethanolamide, preferably in combination with a sucroester, to modify the release of an aliphatic polyamine, preferably spermidine or a salt thereof, more preferably from a composition containing a physiologically acceptable carrier.

Advantageously, the composition of the invention includes, in fact, a physiologically acceptable carrier selected from a cellulose-based compound or a derivative thereof, starches, chitosan, carrageenan, natural rubber, pectin.

Advantageously, the composition described herein allows to make conventional systemic dosage forms with modified release without having to resort to additional release techniques, such as gastro-resistant films or matrix systems based on the use of hydrocolloids.

This advantage is appreciable in the production of food supplements, where the creation of modified release systems is regulated by more binding regulations as compared to the production of drugs, and for example provide limitations in the use of:
coating polymers or solvents that may be used in the pharmaceutical sector
cellulose-based polymers, such as cellulose acetate phthalate, cellulose acetate butyrate, hydroxypropyl methyl cellulose phthalate (HPMCP), hydroxypropyl methyl cellulose acetate succinate (HPMCAS).
acrylic-based polymers, such as functionalized acrylic and methacrylic acid polymers and polymethacrylates for which there are legislative constraints in the case of non-pharmaceutical pre-dosed forms.

According to certain embodiments, the polyamine modified release composition may be incorporated into a composition, preferably for oral administration.

A further subject matter of the invention is therefore a composition, preferably for oral administration, comprising a modified release composition of a biologically active polyamine, preferably spermidine or a salt thereof, according to any one of the embodiments described herein.

The composition for oral administration described herein can be or can be produced in any form suitable for oral administration, such as in the form of a capsule, tablet, dispersion, powder, granules, gummy jellies, freeze-dried discoids.

This composition can be a drug, a nutraceutical, a supplement, preferably for oral administration, for example a food or dietary supplement.

According to certain aspects, the invention relates to the use of palmitoylethanolamide, preferably in combination with a sucroester, for modifying the release of an aliphatic polyamine from a composition containing an aliphatic polyamine, preferably spermidine or a salt thereof.

A further aspect of the invention concerns the non-therapeutic, preferably cosmetic, use of a composition to improve an external appearance of the hair or adnexa thereof, and/or to promote the growth of the hair or adnexa thereof, wherein said composition comprises a composition for the modified release of a biologically active polyamine, comprising a biologically active polyamine, preferably spermidine or a salt thereof, palmitoylethanolamide and a sucroester as substances for modifying the release, and a physiologically acceptable carrier.

Preferably the composition is for oral administration.

The scope of the non-therapeutic, preferably cosmetic uses of the composition, comprises non-therapeutic treatments to improve an external aspect of the hair or adnexa thereof, such as gloss, size of the shaft, in particular its thickness and/or compactness. Furthermore, the non-therapeutic treatment described herein reduces the hair shaft thinning, promotes its physiological growth, and prevents and/or reduces hair loss.

A further aspect of the invention concerns a composition for use in preventing or treating loss of hair or adnexa thereof, comprising a composition for the modified release of a biologically active aliphatic polyamine comprising a biologically active aliphatic polyamine, a substance for modifying the polyamine release and a physiologically acceptable carrier, wherein the release modifying substance comprises palmitoylethanolamide and preferably a sucroester as defined herein.

The composition for medical uses of the invention is suitable for the treatment of scalp and hair growth diseases such as alopecia, androgenetic alopecia, telogenic effluvium; Lichen Planopilaris and forms thereof, alopecia areata, folliculitis decalvans.

Some aspects and advantages of the invention are further described with reference to the attached FIGURE.

### BRIEF DESCRIPTION OF THE FIGURE

The accompanying FIGURE shows a graph relating to the kinetics of spermidine release from five compositions, each containing spermidine, as illustrated in Example 5 where:
PSA means PEA, Spermidine 3 HCl, Starch
SASH5 means Spermidine 3 HCl. Starch, Sucroester HLB 5
SASHA16 means Spermidine 3 HCl, Starch, Sucroester HLB 16
PSASH5 means PEA, Spermidine 3 HCl, Starch, Sucroester HLB 5
PSASH16 means PEA, Spermidine 3 HCl, Starch, Sucroester HLB 16

The graph shows that the compositions that release spermidine more slowly are PSASH5 and PSASH16 according to the preferred and advantageous form of the invention.

### DETAILED DESCRIPTION OF THE INVENTION

According to a first aspect, the invention relates to a composition for the modified release of a biologically active aliphatic polyamine comprising a biologically active aliphatic polyamine, at least a substance for modifying the polyamine release, and a physiologically acceptable carrier, wherein the release modifying substance comprises palmitoylethanolamide.

In the context of the invention, the terms “modified release”, “controlled release” and “extended release” are to be understood as synonymous and interchangeable with each other.

According to a preferred embodiment, the composition for the modified release of a biologically active polyamine comprises palmitoylethanolamide in combination with a sucroester for modifying the aliphatic polyamine release.

Suitable biologically active aliphatic polyamines include putrescine, spermine, spermidine and mixtures thereof, of which spermidine is preferred.

The spermidine, N-(3-aminopropyl)butane-1,4-diamine, contained in the composition can be in the form of a free base or salt, such as spermidine trihydrochloride, i.e. N-(3-aminopropyl)butane-1,4-diamine·3HCl, CAS (Chemical Abstracts Service) Number=334-50-9.

In the context of the present description, the term spermidine is to be understood as including also salts thereof.

Other sources of spermidine are represented by plant matter, which, if appropriately treated using traditional and innovative/modern extraction techniques, can be used to produce spermidine extracts of various strengths.

One of the composition components that contributes to the modified release of the polyamine is palmitoylethanolamide (PEA) or N-(2-hydroxyethyl)hexadecanamide, an endogenous fatty acid amide.

For the purposes of the present invention, palmitoylethanolamide acts as an excipient and/or co-formulant to modulate spermidine release.

Palmitoylethanolamide is well tolerated and free of side effects in animals and humans. The poor solubility of PEA in water, and in gastric simulant and buffer pH 6.8 has been used to counterbalance the high solubility of spermidine, and therefore to create mixed PEA-spermidine systems that allow to modulate the release of spermidine.

Preferably, the palmitoylethanolamide of the composition is not in a ultramicronized form or has an average particle size greater than 6 micrometers.

Advantageously, the composition described herein contains a sucroester, a hydrophilic polymer capable of modulating the release of biogenic polyamines such as spermidine.

In the context of the invention, the term sucroester means esters of sucrose with either saturated, unsaturated, or polyunsaturated fatty acids.

Sucroesters originate from the esterification of sucrose with single fatty acids or mixtures thereof, or from esters thereof in the case of transesterification.

According to some embodiments, the fatty acids are saturated fatty acids with a number of carbon atoms ranging from 4 to 32, preferably selected from lauric acid, myristic acid, palmitic acid, stearic acid, arachidic acid and behenic acid, or they are unsaturated fatty acids with a number of carbon atoms from 14 to 24, and they are preferably palmitoleic acid, oleic acid, erucic acid, or polyunsaturated acids such as linoleic acid, linolenic acid and others.

In a particular embodiment of the invention, the sucroester is an ester of sucrose with nervonic acid.

Sucroesters originate from the esterification of sucrose with single fatty acids or mixtures thereof, or from esters thereof in the case of transesterification.

As regards the degree of esterification, for the purposes of the present invention the following can be used: monoesters, diesters, triesters, or higher esters and combinations thereof.

Typically, the sucroesters used herein, being amphiphilic substances, can be classified on the basis of the hydrophilic/lipophilic balance.

This hydrophilic/lipophilic balance, whose acronym is HLB, allows each raw material to be assigned a score comprised between 0 and 20, where zero coincides with a fully lipophilic substance, while 20 coincides with complete hydrophilicity.

In one embodiment of the invention the HLB of the fatty acids sucrose esters is comprised between 4 and 18.

In one embodiment of the invention the HLB of the fatty acids sucrose esters is comprised between 14 and 16.

In one embodiment of the invention the HLB of the fatty acids sucrose esters is comprised between 5 and 7.

In a particular embodiment of the invention the HLB is about 6 and originates from the use of sucroesters in which the fatty acids are a mixture of stearic and palmitic acid in a ratio of about 60-80/40-20, wherein the monoester percentage is about 30%, the diester percentage is about 50% and higher esters percentage is about 20%.

Examples of suitable sucroesters are:

Sucrose laurate, sucrose myristate, sucrose palmitate, sucrose stearate, sucrose behenate, sucrose oleate, sucrose ricinoleate, sucrose dilaurate, sucrose distearate; sucrose tetraisostearate, sucrose tribehenate, sucrose trilaurate, sucrose tristearate, sucrose palmitate/stearate and mixtures thereof.

For example, sucroesters can be prepared from sucrose and methyl and ethyl esters of fatty acids or by extraction from sucroglycerides using organic solvents such as dimethylformamide, formamide, dimethyl sulfoxide, ethyl acetate, isopropanol, propylene glycol, isobutanol and methyl ethyl ketone.

A suitable physiologically acceptable carrier that can be used in the formulation of the modified release composition described herein is a hydrophilic polymer, preferably selected from the following:
cellulose and derivatives thereof, for example carboxy methyl cellulose, hydroxypropyl methyl cellulose, hydroxypropyl cellulose, methyl cellulose, ethyl cellulose, agar-agar, alginic acid and salts thereof, guar gum, starch, modified and pregelatinized starches, beta-glucans, carrageenans (for example, kappa, iota and lambda and mixtures thereof), gellan, locust bean gum (E410), Konjac gum or Konjac glucomannan (E425), bamboo fiber, inulin, polydextrose, resistant maltodextrin, potato fiber, pea fiber, gum arabic, gum tragacanth, xanthan gum, chitosan, pectins.

Preferred physiologically acceptable carriers are polymers selected from carboxymethylcellulose, corn starch, chitosans for example from Agaricus bisporus, carrageenan, gum arabic, pectin and mixtures thereof.

According to a more preferred embodiment, the modified release composition contains spermidine or a salt thereof, palmitoylethanolamide, sucrose esters of fatty acids (sucroester) and a physiologically acceptable carrier, preferably corn starch.

The modified-release composition described herein releases the active ingredients slowly or in small, repeated amounts over time, for example over a period of 8 hours or more. This form of administration is called modified release, controlled release, sustained release or extended (over time) release. For the purposes of the present invention, these terms are to be considered synonyms.

The modified release composition described herein can be used for the formulation or production of a composition for oral administration containing biologically active substances or pharmacologically active ingredients, and optionally physiologically acceptable carriers.

A further object of the invention are therefore compositions, for example suitable for oral administration, comprising biologically active substances, physiologically acceptable carriers, and a composition for the modified release of a biologically active polyamine comprising an aliphatic polyamine, in particular spermidine or a salt thereof, palmitoylethanolamide and preferably a sucroester.

In certain embodiments, these compositions comprise additional biologically active substances or active ingredients, such as vitamins, minerals, micronutrients and other biologically active substances or extracts of plant origin.

For example, the composition may include one or more vitamins, in particular B vitamins such as B2, B3, B5, B6, B12, vitamin A, vitamin C, vitamin D, in particular D3.

According to some embodiments, the composition of the invention further includes one or more micronutrients and/or minerals such as salts of Zn, Mg, K, Na, Fe, Cr, Se, Mn, Ca, and related mixtures.

According to some embodiments of the invention, the composition may contain probiotic microorganisms in the form of live, viable or inactivated (e.g. tyndallized) cells.

According to some embodiments of the invention, the composition may contain probiotic substances Lacticaseibacillus spp.; Lactiplantibacillus spp.; Levilactobacillus spp. Ligilactobacillus spp.; Limosilactobacillus spp.; Lactobacillus spp.; Bifidobacterium spp.; Bacillus spp., Propionibacterium spp., Saccharomyces spp. Akkermansia spp., Faecalibacterium spp. and Streptococcus spp., and mixtures thereof.

In a particular embodiment, the invention contains Akkermansia muciniphila and Faecalibacterium prausnitzii.

According to some embodiments of the invention, the composition may contain soluble and insoluble prebiotic fibers or in general substances with prebiotic action, alone or in association with probiotic microorganisms. These formulations are generally called symbiotic.

The composition of the invention can take a wide variety of preparation forms, according to the desired route of administration.

The composition of the invention can be in solid form.

When the composition of the invention is presented in solid form, it can be in the form of a tablet, freeze-dried discoids, hard and soft capsules (softgels), lozenges, gummy jellies (gummies), powder, granules, pill to be dissolved in the mouth, and powder or solution/suspension.

The preparations in solid form may include one or more excipients, such as for example starches, sugars, microcrystalline cellulose and optionally diluents, granulating agents, lubricants, aggregating agents, disintegrating agents.

Typically, the composition in solid form may contain an aggregating agent such as gum tragacanth, polyvinylpyrrolidone, gum, corn starch or gelatin; excipients such as dicalcium phosphate; a disintegrating agent such as corn starch, potato starch, alginic acid, super-disintegrants such as crospovidone, cross-linked sodium carboxymethyl cellulose; a lubricant such as magnesium stearate; a sweetening agent such as sucrose, lactose, or saccharin. If desired, the tablets may be coated using traditional techniques.

In some embodiments, the composition is a supplement in hard capsule form, for example gelatin or hydroxypropyl methyl cellulose, which preferably may contain an anti-caking agent, for example, mono- and diglycerides of fatty acids, Vegetable Magnesium Stearate, Silicon Dioxide, and one or more bulking agents such as dibasic calcium phosphate, microcrystalline cellulose.

In some embodiments, the composition of the invention further comprises one or more additional components such as additives, bulking agents or diluents, stabilizers, emulsifiers, texturizers, film forming agents, plasticizers, wetting agents, and thickeners. In the composition there may also be flavoring agents, preservatives, coloring agents, and the like.

In some embodiments, the composition of the invention comprises as an excipient a hydrogenated fatty acid, preferably having a chain having from 3 to 20 carbon atoms, from 14 to 18 carbon atoms. A typical example of a hydrogenated fatty acid is hydrogenated palm oil.

The compositions may be suitably presented in a single pharmaceutical form and prepared using any of the methods well known in the pharmaceutical or dietetic state of the art.

In some embodiments, in the compositions of the present invention, the active ingredients are normally formulated in a dosage unit. The dosage unit may contain from 0.001 to 1.000 mg of each active ingredient per dosage unit for a daily administration.

In some embodiments, the dose is in the range from 0.001 wt % to about 60 wt % of the formulation.

As used herein, the term “carrier” means a medium, excipient, diluent with which the combination of therapeutic or active ingredients is administered.

Any carrier and/or excipient suitable for the desired form of preparation for administration to humans is contemplated for use with the compounds described in the present invention.

As used herein, the term combination means that one or more of the active ingredients are added to or mixed with one or more ingredients. The term combination is not intended to mean that the active ingredients are associated with each other through the formation of chemical or other bonds.

The compositions of the invention are suitable for food, nutritional, dietary, or pharmaceutical use in mammals, particularly in humans.

According to some embodiments, the composition of the invention is contained in a food supplement, a nutritional product, or a dietary product.

In some embodiments the composition, or the supplement containing it, is administered once or twice a day.

Advantageously, the compositions described herein find application in the trichology sector, for example in favoring the conditions for the growth of the keratin structures of a mammal such as hair, eyebrows, eyelashes, and beard.

These activities are promoted by the presence of spermidine in the composition, in particular by its prolonging effect on the anagen phase of the hair life cycle and by the stimulation of the hair follicle stem cells.

In the context of the present description, the combination of PEA and spermidine may also be referred to as the PEA-Spermidine complex or matrix.

For the controlled release of aliphatic polyamines, especially, spermidine will be more apparent from the following examples. In these examples, the polyamine/spermidine controlled release composition is also referred to as the matrix.

### Example 1

Composition for the modified release of spermidine having the following formulation expressed as a percentage by weight:

| | |
|---|---|
| Spermidine trihydrochloride | 10% |
| Palmitoylethanolamide (PEA) | 10% |
| Sucrose esters of fatty acids (sucroester) | 10% |
| Carrier: 70% corn starch | 70% |

### Example 2

Composition for oral administration containing the composition for the modified release of spermidine in combination with additional active ingredients and excipients, having the following formulation:

COATED TABLET

| | |
|---|---|
| Ingredient | amount/tablet |

| | | |
|---|---|---|
| Spermidine retard (corn starch, sucrose | 5.5 | mg |
| esters of fatty acids, N-(3-aminopropyl)- | | |
| tetramethylenediamine trihydrochloride, | | |
| Palmitoylethanolamide | | |
| For a total of | 0.55 | mg |
| Borage Seed Oil Powder [Borage | 126 | mg |
| (Borrago Officinalis L.), | | |
| Tapioca dextrins, silicon dioxide] | | |
| For a total of Oil rich in unsaturated | 60 | mg |
| omega 3, 6, 9 fatty acids | | |
| Star anise dry extract, 20% shikimic | 100 | mg |
| acid (Illicium verum Hook fruit dry | | |
| extract, Maltodextrin) | | |
| For a total of shikimic acid | 20 | mg |
| Reishi (Ganoderma lucidum - Curtis- | 70 | mg |
| P. Karst.) sporophore dry extract | | |
| titrated at 20% polysaccharides | | |
| For a total of Reishi polysaccharides | 14 | mg |
| Low viscosity hydroxypropyl methylcellulose | 16 | mg |
| Dicalcium phosphate | 160 | mg |
| Croscarmellose Sodium | 10.0 | mg |
| Rutin | 10.33 | mg |
| Zinc bisglycinate | 26 | mg |
| For a total content of zinc | 7.5 | mg |
| Sacred basil [(Ocimum sanctum L.) | 100.0 | mg |
| leaves dry extract, maltodextrin, | | |
| silicon dioxide] | | |
| For a total content of ursolic acid | 2 | mg |
| Microcrystalline cellulose | 233.40 | mg |
| Silicon dioxide | 8.6 | mg |
| Mono- and diglycerides of fatty acids | 12.8 | mg |
| Clear coating (hydroxypropyl | 26 | mg |
| methylcellulose, stearic acid, | | |
| microcrystalline cellulose) | | |

### Example 3

### Preparation of a Composition in the Form of Capsule for Dissolution Test:

| | | | | | | | Gross Weight | |
|---|---|---|---|---|---|---|---|---|
| | | Composition or | | Type 1 | | | (capsule + | |
| | | matrix for controlled | | Capsule | Net | Net | powder) from | |
| | | release of PEA- | SERIES | Weight | Weight | Weight | scale | |
| SERIES | No. | Spermidine | No. | (g) | (g) | (mg) | (g) | Δ |
| 1 | 1 | PEA-SPE-CHI -SUC | 1 | 0.0781 | 0.1014 | 101.4 | 0.1795 | 0.0000 |
| | 2 | PEA-SPE-CHI-SUC | 2 | 0.0804 | 0.1004 | 100.4 | 0.1807 | 0.0001 |
| | 3 | PEA-SPE-CHI-SUC | 3 | 0.0778 | 0.0986 | 98.6 | 0.1764 | 0.0000 |
| 2 | 4 | PEA-SPE-CAR-SUC | 1 | 0.0769 | 0.1014 | 101.4 | 0.1786 | −0.0003 |
| | 5 | PEA-SPE-CAR-SUC | 2 | 0.0801 | 0.1016 | 101.6 | 0.1817 | 0.0000 |
| | 6 | PEA-SPE-CAR-SUC | 3 | 0.0775 | 0.1 | 100 | 0.1775 | 0.0000 |
| 3 | 7 | PEA-SPE-PEC-SUC | 1 | 0.0768 | 0.0996 | 99.6 | 0.1762 | 0.0002 |
| | 8 | PEA-SPE-PEC-SUC | 2 | 0.074 | 0.0999 | 99.9.9 | 0.1739 | 0.0000 |
| | 9 | PEA-SPE-PEC-SUC | 3 | 0.0785 | 0.1016 | 101.6 | 0.1802 | −0.0001 |
| 4 | 10 | PEA-SPE-AMI-SUC | 1 | 0.0775 | 0.1011 | 101.1 | 0.1787 | −0.0001 |
| | 11 | PEA-SPE-AMI-SUC | 2 | 0.0767 | 0.1002 | 100.2.2 | 0.1768 | 0.0001 |
| | 12 | PEA-SPE-AMI-SUC | 3 | 0.0799 | 0.0992 | 99.2.2 | 0.179 | 0.0001 |
| 5 | 13 | PEA-SPE-GAR-SUC | 1 | 0.0764 | 0.1012 | 101.2.2 | 0.1775 | 0.0001 |
| | 14 | PEA-SPE-GAR-SUC | 2 | 0.0777 | 0.1015 | 101.5 | 0.1792 | 0.0000 |
| | 15 | PEA-SPE-GAR-SUC | 3 | 0.079 | 0.0996 | 99.6 | 0.1786 | 0.0000 |
| 6 | 16 | PEA-SPE-CMC-SUC | 1 | 0.0816 | 0.1005 | 100.5 | 0.1822 | −0.0001 |
| | 17 | PEA-SPE-CMC-SUC | 2 | 0.0825 | 0.1018 | 101.8 | 0.1842 | 0.0001 |
| | 18 | PEA-SPE-CMC-SUC | 3 | 0.0756 | 0.1007 | 100.7.7 | 0.1757 | 0.0006 |

The term matrix is used to identify the modified release composition.

### Dissolution Assay for Capsules:

Dissolution apparatus with basket
Test duration: 8 hours; medium temperature: 37° C.; shaft rotation speed: 100 rpm 2 h in 0.1 N HCl solution, and subsequent addition of phosphate buffer pH 6.8 for up to 8 hours.

The following procedure was adopted:

Introduce 750 ml of 0.1N HCl into each of the containers. Start stirring the baskets at 100 rpm and wait for the temperature of the medium to stabilize at 37° C.±0.5° C.

2 hours after starting the test, add 200 ml, carefully measured, of 0.2 M Na₃PO₄*12H₂O previously thermostated at 37° C. to each of the 6 containers, adjust the pH to 6.8 with 2N NaOH, if necessary.

Carry out sampling after, 2 h, 4 h, 6 h, and 8 h.

### Results

| | | | mg | mg | | | |
|---|---|---|---|---|---|---|---|
| API | CODE | DESCRIPTION | RELEASED | THEORICAL | RECOVERY | AVERAGE | RSD |
| Spermidine | Blank | | 0 | | | | |
| Spermidine | PH-22-022115 | PEA-SPE-GAR -SUC complex | 10.95 | 10.00 | 109 | 104 | 7.8 |
| | 2 h A | | | | | | |
| Spermidine | PH-22-022116 | PEA-SPE-CAR-SUC complex | 2.92 | 10.00 | 29 | 29 | 2.0 |
| | 2 h A | | | | | | |
| Spermidine | PH-22-022117 | PEA-SPE-PEC-SUC complex | 9.76 | 10.00 | 98 | 96 | 2.0 |
| | 2 h A | | | | | | |
| Spermidine | PH-22-022118 | PEA-SPE-CHI-SUC complex | 7.94 | 10.00 | 79 | 73 | 11.4 |
| | 2 h A | | | | | | |
| Spermidine | PH-22-022119 | PEA-SPE-CMC-SUC complex | 8.58 | 10.00 | 86 | 83 | 5.0.0 |
| | 2 h A | | | | | | |
| Spermidine | PH-22-022120 | PEA-SPE-AMI-SUC complex | 8.07 | 10.00 | 81 | 80 | 1.6 |
| | 2 h A | | | | | | |
| Spermidine | PH-22-022115 | PEA-SPE-GAR-SUC complex | 9.80 | 10.00 | 98 | | |
| | 2 h B | | | | | | |
| Spermidine | PH-22-022116 | PEA-SPE-CAR-SUC complex | 2.84 | 10.00 | 28 | | |
| | 2 h B | | | | | | |
| Spermidine | PH-22-022117 | PEA-SPE-PEC-SUC complex | 9.49 | 10.00 | 95 | | |
| | 2 h B | | | | | | |
| Spermidine | PH-22-022118 | PEA-SPE-CHI-SUC complex | 6.75 | 10.00 | 68 | | |
| | 2 h B | | | | | | |
| Spermidine | PH-22-022119 | PEA-SPE-CMC-SUC complex | 7.99 | 10.00 | 80 | | |
| | 2 h B | | | | | | |
| Spermidine | PH-22-022120 | PEA-SPE-AMI-SUC complex | 7.89 | 10.00 | 79 | | |
| | 2 h B | | | | | | |
| Spermidine | PH-22-022115 | PEA-SPE-GAR-SUC complex | 13.00 | 10.00 | 130 | 113 | 21.5 |
| | 4 h A | | | | | | |
| Spermidine | PH-22-022116 | PEA-SPE-CAR-SUC complex | 4.14 | 10.00 | 41 | 43 | 6.2 |
| | 4 h A | | | | | | |
| Spermidine | PH-22-022117 | PEA-SPE-PEC-SUC complex | 11.10 | 10.00 | 111 | 115 | 5.2 |
| | 4 h A | | | | | | |
| Spermidine | PH-22-022118 | PEA-SPE-CHI-SUC complex | 9.47 | 10.00 | 95 | 80 | 26.7 |
| | 4 h A | | | | | | |
| Spermidine | PH-22-022119 | PEA-SPE-CMC-SUC complex | 11.32 | 10.00 | 113 | 95 | 26.5 |
| | 4 h A | | | | | | |
| Spermidine | PH-22-022120 | PEA-SPE-AMI-SUC complex | 9.31 | 10.00 | 93 | 94 | 0.7 |
| | 4 h A | | | | | | |
| Spermidine | PH-22-022115 | PEA-SPE-GAR-SUC complex | 9.57 | 10.00 | 96 | | |
| | 4 h B | | | | | | |
| Spermidine | PH-22-022116 | PEA-SPE-CAR-SUC complex | 4.52 | 10.00 | 45 | | |
| | 4 h B | | | | | | |
| Spermidine | PH-22-022117 | PEA-SPE-PEC-SUC complex | 11.94 | 10.00 | 119 | | |
| | 4 h B | | | | | | |
| Spermidine | PH-22-022118 | PEA-SPE-CHI-SUC complex | 6.46 | 10.00 | 65 | | |
| | 4 h B | | | | | | |
| Spermidine | PH-22-022119 | PEA-SPE-CMC complex | 7.74 | 10.00 | 77 | | |
| | 4 h B | | | | | | |
| Spermidine | PH-22-022120 | PEA-SPE-AMI-SUC complex | 9.40 | 10.00 | 94 | | |
| | 4 h B | | | | | | |
| Spermidine | PH-22-022115 | PEA-SPE-GAR-SUC complex | 12.43 | 10.00 | 124 | 102 | 30.4 |
| | 6 h A | | | | | | |
| Spermidine | PH-22-022116 | PEA-SPE-CAR-SUC complex | 2.33 | 10.00 | 23 | 30 | 30.5 |
| | 6 h A | | | | | | |
| Spermidine | PH-22-022117 | PEA-SPE-PEC-SUC complex | 11.41 | 10.00 | 114 | 103 | 15.6 |
| | 6 h A | | | | | | |
| Spermidine | PH-22-022118 | PEA-SPE-CHI-SUC complex | 9.51 | 10.00 | 95 | 96 | 0.7 |
| | 6 h A | | | | | | |
| Spermidine | PH-22-022119 | PEA-SPE-CMC-SUC complex | 9.99 | 10.00 | 100 | 95 | 7.1 |
| | 6 h A | | | | | | |
| Spermidine | PH-22-022120 | PEA-SPE-AMI-SUC complex | 7.49 | 10.00 | 75 | 82 | 11.7 |
| | 6 h A | | | | | | |
| Spermidine | PH-22-022115 | PEA-SPE-GAR-SUC complex | 8.04 | 10.00 | 80 | | |
| | 6 h B | | | | | | |
| Spermidine | PH-22-022116 | PEA-SPE-CAR-SUC complex | 3.61 | 10.00 | 36 | | |
| | 6 h B | | | | | | |
| Spermidine | PH-22-022117 | PEA-SPE-PEC-SUC complex | 9.14 | 10.00 | 91 | | |
| | 6 h B | | | | | | |
| Spermidine | PH-22-022118 | PEA-SPE-CHI-SUC complex | 9.60 | 10.00 | 96 | | |
| | 6 h B | | | | | | |
| Spermidine | PH-22-022119 | PEA-SPE-CMC-SUC complex | 9.03 | 10.00 | 90 | | |
| | 6 h B | | | | | | |
| Spermidine | PH-22-022120 | PEA-SPE-AMI-SUC complex | 8.84 | 10.00 | 88 | | |
| | 6 h B | | | | | | |
| Spermidine | PH-22-022115 | PEA-SPE-GAR-SUC complex | 12.06 | 10.00 | 121 | 117 | 4.7 |
| | 8 h A | | | | | | |
| Spermidine | PH-22-022116 | PEA-SPE-CAR-SUC complex | 4.27 | 10.00 | 43 | 42 | 1.2 |
| | 8 h A | | | | | | |
| Spermidine | PH-22-022117 | PEA-SPE-PEC-SUC complex | 12.62 | 10.00 | 126 | 120 | 7.0 |
| | 8 h A | | | | | | |
| Spermidine | PH-22-022118 | PEA-SPE-CHI-SUC complex | 9.48 | 10.00 | 95 | 94 | 0.5 |
| | 8 h A | | | | | | |
| Spermidine | PH-22-022119 | PEA-SPE-CMC-SUC complex | 11.77 | 10.00 | 118 | 109 | 10.7 |
| | 8 h A | | | | | | |
| Spermidine | PH-22-022120 | PEA-SPE-AMI-SUC complex | 10.77 | 10.00 | 108 | 112 | 5.6 |
| | 8 h A | | | | | | |
| Spermidine | PH-22-022115 | PEA-SPE-GAR-SUC complex | 11.2.29 | 10.00 | 113 | | |
| | 8 h B | | | | | | |
| Spermidine | PH-22-022116 | PEA-SPE-CAR-SUC complex | 4.20 | 10.00 | 42 | | |
| | 8 h B | | | | | | |
| Spermidine | PH-22-022117 | PEA-SPE-PEC-SUC complex | 11.44 | 10.00 | 114 | | |
| | 8 h B | | | | | | |
| Spermidine | PH-22-022118 | PEA-SPE-CHI-SUC complex | 9.41 | 10.00 | 94 | | |
| | 8 h B | | | | | | |
| Spermidine | PH-22-022119 | PEA-SPE-CMC-SUC complex | 10.12 | 10.00 | 101 | | |
| | 8 h B | | | | | | |
| Spermidine | PH-22-022120 | PEA-SPE-AMI-SUC complex | 11.66 | 10.00 | 117 | | |
| | 8 h B | | | | | | |

### Example 4

### Preparation of Compositions Based on a PEA-Spermidine Combination

### Study Objective

The present study aimed to prepare combinations, referred to as complexes, between palmitoylethanolamide (PEA) and spermidine trihydrochloride (SPE) on various carriers, with the aim of obtaining a controlled release of spermidine from the same carrier.

### Materials and Methods

### Active Principle

SPE—Spermidine trihydrochloride

### Co-Formulating Ingredient, Suitable for Modified Release

PEA—Palmitoylethanolamide,

### Agent for Release Modulation

SUC—Sucroesters or Esters of Sucrose E473,

### Emulsifier

MGE—Mono and diglycerides of fatty acids E471,

### Carrier

CMC—Carboxymethylcellulose E466,
AMI—Corn starch,
CHI—Vegetable chitosan from Agaricus bisporus
CAR—Carrageenan,
GAR—Gum arabic,
PEC—Pectin E440,

### Solvents

H₂O—Distilled water
EtOH—95% vol Ethanol (alcohol).

### Solubility Evaluation

First of all, an evaluation of the solubility of PEA and SPE in water and ethanol was carried out in the presence of two different concentrations of the two emulsifiers.

Data known from the literature:
SPE

MW=145.25 g/mol; solubility in water=50 g/l
PEA

MW=299.49 g/mol; solubility in water=insoluble; solubility in ethanol: 16 g/l.

The tests were carried out as summarized in the following Table 1:

TABLE 1
Preparation of solutions A-H 1

| | 1 | 2 | 3 | 4 | 5 |
|---|---|---|---|---|---|
| | SPE | PEA | H₂O e/o EtOH | SUC | MGE |
| A | 50 mg | — | H₂O q.s. to 1 g | — | — |
| B | — | 50 mg | H₂O q.s. to 1 g | — | — |
| C | — | 50 mg | H₂O q.s. to 5 g | — | 5 mg |
| D | — | 50 mg | H₂O q.s. to 5 g | 5 mg | — |
| E | — | 50 mg | H₂O q.s. to 5 g | — | 50 mg |
| F | — | 50 mg | H₂O q.s. to 5 g | 50 mg | — |
| G | — | 32 mg | EtOH q.s. to 2 g | — | — |
| H | 50 mg | 50 mg | EtOH q.s. to 2.5 g, | 50 mg | |
| | | | then H₂O q.s. to 3 g | | |
| | 1 | 2 | 3 | 4 | 5 |
| | SPE | PEA | H₂O and/or EtOH | SUC | MGE |
| A | 50 mg | — | H₂O q.s. to 1 g | — | — |
| B | — | 50 mg | H₂O q.s. to 1 g | — | — |
| C | — | 50 mg | H₂O q.s. to 5 g | — | 5 mg |
| D | — | 50 mg | H₂O q.s. to 5 g | 5 mg | — |
| E | — | 50 mg | H₂O q.s. to 5 g | — | 50 mg |
| F | — | 50 mg | H₂O q.s. to 5 g | 50 mg | — |
| G | — | 32 mg | EtOH q.s. to 2 g | — | — |
| H | 50 mg | 50 mg | EtOH q.s. to 2.5 g, | 50 mg | |
| | | | then H₂O q.s. to 3 g | | |

In test A, the solubility of SPE in water (50 mg/ml) was confirmed.

In test B the insolubility of PEA in water was confirmed.

In test C, the addition of the emulsifier MGE in a ratio equal to PEA/MGE=10:1 w/w does not seem to improve the solubility of PEA.

In test D, the addition of the emulsifier SUC in a ratio equal to PEA/SUC=10:1 w/w does not seem to improve the solubility of PEA; however, some clouding of the liquid phase appears.

In both test C and test D, water was added initially up to 1 ml, then to 2 ml and finally to 5 ml. Hot solubility does not appear to have significant improvements.

Test E is similar to C, but the PEA/MGE ratio is equal to 1:1 w/w. The result is comparable to test C.

Test F is similar to D, but the PEA/SUC ratio is equal to 1:1 w/w. Solubility appears to increase slightly, but not sufficiently.

In test G, the solubility of PEA in ethanol (16 mg/ml) was confirmed.

In test H, 50 mg of PEA (crushed in the mortar because it was initially in the form of flakes) was placed in a 10 ml test tube and 50 mg of SUC were added.

95% vol EtOH was added in an amount equal to the solubility of PEA (3.125 ml of EtOH, equal to about 2.5 g, for 50 mg of PEA). After heating, the mixture became a perfectly clear solution. 300 mg of water were then added. At the time of addition, cloudiness was observed, but when heated it returned to being a clear solution again. Another 200 mg of water were then added, and the same behavior was observed. This time, it was necessary to heat for a longer time to obtain a clear solution again. Finally, 50 mg of SPE (PEA/SPE ratio=1:1 w/w) were added, which dissolved immediately.

### Note:

1) PEA was initially present in the form of flakes. It was reduced in size in the mortar before testing.

2) Emulsifiers were added to PEA before adding solvents.

### Results

The solution of test H was selected for the subsequent tests for the formation of PEA-SPE combinations (complexes).

### Preparation of Combinations/Complexes Using the Solvent Evaporation Method “Kneading Technique”

100 g of solution H were initially prepared according to the procedure described above, using:
1.67 g of SPE
1.67 g of PEA
1.67 g of SUC
78.5 g of 95% vol EtOH.
16.5 g of H₂O.

2.5 g of each of the six (6) complexes were prepared. Each combination (complex) includes Carrier+SPE+PEA+SUC in the following w/w proportions: 70% carrier, 10% SPE, 10% PEA, 10% SUC. For each combination, 1.75 g of carrier and 15 g of solution H were used (15 g of solution contains 250 mg of SPE, 250 mg of PEA and 250 mg of SUC).

1.75 g of carrier were placed in a crystallizer, to which 15 g of hot solution were added in small aliquots. After each addition, the mixture was kneaded with a spatula. At the end of the additions, the crystallizers were placed in a ventilated oven at 50° C. overnight, until the solvents had completely evaporated. These operations were repeated for each combination, each in a separate crystallizer.

### Notes

1) At the end of the addition the mixtures are rather liquid.

2) Solution H must be kept hot in a water bath for the entire duration of the additions.

### Results

Due to the macroscopic characteristics they have, the complexes obtained are all suitable for subsequent tests for the release of the active ingredients from the carriers.

### Conclusion

After the incorporation of the complexes into capsules and tablets, the dissolution capacity and the release properties of the active ingredients from the carriers, the complexes will be prepared at an industrial level using methods such as spray-drying or possibly freeze-drying.

At the end of the study, the following products were provided:
Combination PEA-SPE-CHI-SUC, 2 g
Combination PEA-SPE-AMI-SUC, 2 g,
Combination PEA-SPE-CAR-SUC, 2 g,
Combination PEA-SPE-CMC-SUC, 2 g,
Combination PEA-SPE-GAR-SUC, 2 g,
Combination PEA-SPE-PEC-SUC, 2 g.
   B) Preparation of PEA-SPE-AMI-SUC Complexes by Kneading Technique Using Two Sucroesters with Different HLB Values

### Study Objective

The present study aimed to prepare complexes between spermidine (SPE) and two different sucroesters (SUC) with two different hydrophilic-lipophilic balance values.

### Materials and Methods

### Active Ingredients

SPE—Spermidine.

### Co-Formulants for Sustained Release-Emulsifiers

SUC-HLB16—Sucroesters or Esters of Sucrose E473, hydrophilic-lipophilic balance HLB≈16
SUC-HLB5—Sucroesters or Esters of Sucrose E473, hydrophilic-lipophilic balance HLB≈5

### Carrier

AMI—Corn starch

### Solvents

H₂O—Distilled water
EtOH—95% vol Ethanol (alcohol).

### Preparation of the Complexes Using the Solvent Evaporation Method “Kneading Technique”

Two solutions were initially prepared, one for each of the two sucroesters. The solutions were prepared as described in the previous procedure for test H, using:
3.00 g of SPE
3.00 g of SUC (HLB5 or HLB16)
140.0 g of 95% vol EtOH.
30.0 g of H₂O.

Subsequently, each of the two solutions was poured into a crystallizer, after which the crystallizers were placed in a ventilated oven at 50° C. overnight, until the solvents had completely evaporated.

### Results and Conclusions

The following products were supplied:
Combination SPE-SUC-HLB16, 5 g,
Combination SPE-SUC-HLB5, 5 g,
   C) Preparation of PEA-SPE-AMI-SUC Complexes by Atomization Using Two Sucroesters with Different HLB Values

### Study Objective

The present study aimed to prepare complexes between palmitoylethanolamide (PEA) and spermidine (SPE) and two different sucroesters (SUC) with two different hydrophilic-lipophilic balance values, on native starch used as a carrier.

### Materials and Methods

### Active Ingredient

SPE—Spermidine,

### Controlled Release Agent

PEA—Palmitoylethanolamide,

### Co-Formulants for Controlled Release—Emulsifiers

SUC-HLB16—Sucroesters or Esters of Sucrose E473 hydrophilic-lipophilic balance HLB≈16
SUC-HLB5—Sucroesters or Esters of Sucrose E473, hydrophilic-lipophilic balance HLB≈5

### Carrier

AMI—Native corn starch

### Solvents

H₂O—Distilled water
EtOH—95% vol Ethanol (alcohol).

### Preparation of Complexes by Atomization

Two solutions were initially prepared, one for each of the two sucroesters. The solutions were prepared as described in the previous procedure for test H.

Subsequently, the two solutions were subjected to atomization (spray-drying) on to starch.

### Results and Conclusions

The following products were supplied:
PEA-SPE-AMI—SUC-HLB16 ATOMIZED Combination, 715 g
PEA-SPE-AMI—SUC-HLB5 ATOMIZED Combination, 700 g
   D) Preparation of SPE-AMI-SUC Complexes by Kneading Technique Using Two Sucroesters with Different HLB Values

### Study Objective

The present study aimed to prepare complexes between spermidine (SPE) and two different sucroesters (SUC) with two different hydrophilic-lipophilic balance values, supported on starch (AMI), and the preparation of a combination between palmitoylethanolamide (PEA) and spermidine (SPE) on starch without the use of sucroester.

### Materials and Methods

### Active Ingredients

SPE—Spermidine,

### Controlled Release Agent

PEA—Palmitoylethanolamide,

### Co-Formulants for Controlled Release—Emulsifiers

SUC-HLB16—Sucroesters or Esters of sucrose E473 hydrophilic-lipophilic balance HLB≈16
SUC-HLB5—Sucroesters or Esters of sucrose E473, hydrophilic-lipophilic balance HLB≈5, provided by Giuliani

### Carrier

AMI—Corn starch,

### Solvents

H₂O—Distilled water
EtOH—95% vol Ethanol (alcohol).

### Preparation of the Complexes Using the Solvent Evaporation Method “Kneading Technique”

Using the previous procedure for test H, 75 g of each of the following three solutions were initially prepared:

| Solution 1) | Solution 2) | Solution 3) |
|---|---|---|
| 1.25 g of SPE | 1.25 g of SPE | 1.25 g of SPE |
| 1.25 g of SUC (HLB5) | 1.25 g of SUC (HLB16) | 1.25 g of PEA |
| 59.0 g of 95% | 59.0 g of 95% | 59.0 g of 95% |
| vol EtOH. | vol EtOH. | vol EtOH. |
| 12.4 g di H₂O | 12.4 g of H₂O | 12.4 g of H₂O |

7 g of starch as carrier were placed in each of three different crystallizers, to which 60 g of each of the three solutions were added hot in small aliquots. After each addition, the mixture was kneaded with a spatula. At the end of the additions, the crystallizers were placed in a ventilated oven at 50° C. overnight, until the solvents had completely evaporated.

### Results and Conclusions

The following products were supplied:
SPE-AMI-SUC-HLB5 combination, 8 g,
SPE-AMI-SUC-HLB16 combination, 8 g,
PEA-SPE-AMI combination, 8 g,

### Example 5

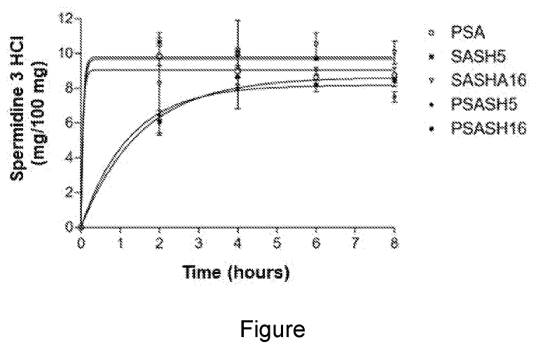

Dissolution tests of the composition/release system of Spermidine (SPE), palmitoylethanolamide (PEA), sucroester were performed. The results are reported in the attached FIG. 1 in which:
PSA means PEA, Spermidine 3 HCl, Starch
SASH5 means Spermidine 3 HCl. Starch, Sucroester HLB 5
SASHA16 means Spermidine 3 HCl, Starch, Sucroester HLB 16
PSASH5 means PEA, Spermidine 3 HCl, Starch, Sucroester HLB 5
PSASH16 means PEA, Spermidine 3 HCl, Starch, Sucroester HLB 16

The kinetics of spermidine release from the various complexes was evaluated using a nonlinear regression model based on a pseudo-first order equation:

$$Y = Y 0 + ( Plateau - Y 0 ) * ( 1 - exp ( - K * x ) )$$

where Y is the amount of spermidine trihydrochloride released expressed in mg/100 mg
X is the time in hours
Y 0 is the amount of spermidine released at time 0 which is 0
Plateau is the maximum amount of spermidine that can be released from a complex (at an infinite time).
K is the kinetic constant and is calculated in the nonlinear regression process

The nonlinear regression model was done using GraphPad Prism 9 Curve Fitting software https://www.graphpad.com.

The complexes showing slower release (modified release) are PSASH5 and PSASH16. Their Half-Life is approximately 45-60 minutes, which is more than 16 times longer than that of other complexes, which release spermidine more rapidly.

1. A composition for the modified release of a biologically active aliphatic polyamine comprising a biologically active aliphatic polyamine, a substance for modifying the release of the polyamine, and a physiologically acceptable carrier, wherein the substance for modifying the release comprises palmitoylethanolamide.

2. The composition according to claim 1, wherein the biologically active aliphatic polyamine is spermidine or a salt thereof.

3. The composition according to claim 1, wherein the substance for modifying the release further comprises a sucroester.

4. The composition according to claim 3, wherein the sucroester has a hydrophilic/lipophilic or HLB balance comprised between 4 and 18.

5. The composition according to claim 1, wherein the physiologically acceptable carrier is selected from a cellulose-based compound, starches, chitosan, carrageenan, natural rubber, pectin, and mixtures thereof.

6. A food supplement for oral administration comprising a composition for the modified release of a biologically active polyamine according to claim 1.

7. A non-therapeutic method for improving an external appearance of hair or cutaneous adnexa thereof and/or for promoting the growth of hair or cutaneous adnexa thereof comprising the step of administering the composition according to claim 1.

8. A method for preventing or treating a disease that causes loss of hair or cutaneous adnexa thereof, wherein said method comprise the step of administering the composition according to claim 1.

9. The method according to claim 8, wherein the treatment occurs by oral administration.

10. The method according to claim 8, wherein said disease causing loss of hair or cutaneous adnexa thereof is selected from the group consisting of alopecia, androgenetic alopecia, telogenic effluvium, Lichen Planopilaris, alopecia areata, and folliculitis decalvans.

11. A method for modifying the release of an aliphatic polyamine from a composition containing a physiologically acceptable carrier comprising the step of administering palmitoylethanolamide.

12. The composition according to claim 2, wherein the biologically active aliphatic polyamine is spermidine trihydrochloride.

13. The composition according to claim 4, wherein the sucroester has a hydrophilic/lipophilic or HLB balance comprised from 14 to 16 or from 5 to 7.

14. The food supplement for oral administration of claim 6, wherein the biologically active polyamine is spermidine or a salt thereof.

15. The non-therapeutic method according to claim 7, wherein the step of administering the composition is carried out by oral administration.

16. The method according to claim 11, wherein the palmitoylethanolamide is used in combination with a sucroester.

17. The method according to claim 11, wherein the aliphatic polyamine is spermidine or a salt thereof.
